(19) United States
(12) Patent Application Publication
(10) Pub. No.: US 2023/0295328 A1
(43) Pub. Date: Sep. 21, 2023

(54) TREATMENT OF IDIOPATHIC INFLAMMATORY MYOPATHIES

(71) Applicant: ADIENNE S.A., Lugano (CH)

(72) Inventor: Antonio Francesco DI NARO, Morcote (CH)

(73) Assignee: ADIENNE S.A., Lugano (CH)

(21) Appl. No.: 18/007,278

(22) PCT Filed: Jul. 28, 2020
(86) PCT No.: PCT/IB2020/057122

§ 371 Date: Jan. 27, 2023

(51) Int. Cl. C07K 16/28; A61K 31/5377; A61K 31/519; A61K 31/52; A61K 31/573; A61K 31/675; A61K 38/13; A61K 31/436; A61K 39/395; A61P 21/00; A61P 29/00

(52) U.S. Cl. CPC C07K 16/2896; A61K 31/436; A61K 31/519; A61K 31/52; A61K 31/5377; A61K 31/573; A61K 31/675; A61K 38/13; A61K 39/3955; A61P 21/00; A61P 29/00; C07K 2317/21; C07K 2317/24; C07K 2317/35

(57) ABSTRACT

The invention provides a method of treating an idiopathic inflammatory myopathy in a human patient by administering an anti-CD26 antibody.

### FIELD OF THE INVENTION

The invention disclosed herein relates to methods of treating an idiopathic inflammatory myopathy with an antibody that specifically binds CD26.

### BACKGROUND OF THE INVENTION

Idiopathic inflammatory myopathy (IIM) is a group of disorders characterized by inflammation of the muscles used for movement (skeletal muscles). The primary symptom is muscle weakness but these myopathies can affect multiple organs apart from muscle and often lead to a severe impairment of the quality of life.

Currently, there are not approved therapies in Europe and the United States for the treatment of certain myopathies, such as dermatomyositis (DM). Glucocorticoids are considered to be the mainstay of initial management (Moghadam-Kia et al, Clin. Rev. Allergy Immunol. 52:81-87 (2017)) being the first-line treatment despite their various adverse effects, delivered topically, orally, or intravenously. Additional immunosuppressive drugs, such as methotrexate, azathioprine and mycophenolate mofetil, may reduce inflammation in patients who do not respond well to prednisone and can be started simultaneously, unless only very mild symptoms are present (Carstens et al, Clin. Exp. Immunol. 175:425-438 (2014)). However, there is no convincing evidence for the efficacy of these agents in myositis. Moreover, methotrexate, azathioprine and mycophenolate mofetil are potentially teratogenic, which makes an effective contraception essential (Makol et al, Drugs 71:1973-1987 (2011)) for women planning to become pregnant.

It is an object of the present invention to provide improved methods for treating IIM.

### SUMMARY OF THE INVENTION

The present invention is directed to a method of inhibiting inflammation in a human patient having an IIM comprising administering to the patient an effective amount of an antibody that specifically binds CD26. The present invention is also directed to a method of treating an (IIM) comprising administering to the patient an effective amount of an antibody that specifically binds CD26. In one embodiment, the IIM is dermatomyositis (DM), polymyositis (PM), necrotizing myopathy (NM), sporadic inclusion body myositis (IBM), or overlap myositis (OM).

In one embodiment, the anti-CD26 antibody is a full-length antibody. In another embodiment, the antibody is a monoclonal, human, humanized, chimeric, multivalent antibody, or an antigen-binding fragment thereof. In another embodiment, the antibody has an isotype selected from the group consisting of IgG1, IgG2, IgG3, IgG4, IgM, IgA, IgD and IgE. In another embodiment, the antibody has an IgG2b isotype. In another embodiment, the anti-CD26 antibody is begelomab, 1F7, or CM03. In another embodiment, the anti-CD26 antibody is produced in Chinese hamster ovary (CHO) cells. In another embodiment, the anti-CD26 antibody is produced from a hybridoma cell line deposited at CBA-ICLC of Genoa (Italy) as deposit number PD 12002.

In another embodiment, the anti-CD26 antibody comprises (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12. In one embodiment, the anti-CD26 antibody comprises heavy and light chain variable regions comprising the sequences set forth in SEQ ID NOs:3 and 5, respectively. In one embodiment, the anti-CD26 antibody comprises heavy and light chains constant regions comprising the sequences set forth in SEQ ID NOs: 1 and 2, respectively.

In one embodiment the method further comprises administration of an immunosuppressive agent. In one embodiment, the immunosuppressive agent is a corticosteroid, methotrexate, azathioprine, mycophenolate mofetil, methylprednisone, cyclophosphamide, cyclosporineA, tacrolimus, or a combination thereof. In one embodiment, the immunosuppressive agent is given prior to, concomitantly with, or after the anti-CD26 antibody.

In one embodiment, the administration cycle is a period of 4 or 5 weeks, and wherein for each of the at least one cycle, 16 doses of the anti-CD26 antibody are administered at a dose of between 4.0 mg/m² to 25 mg/m². In one embodiment, the anti-CD26 antibody is administered at a dose of 4, 5, 6, 7, 8, 9, 10, 11, 12, 13, 14, 15, 16, 17, 18, 19, 20, 21, 22, 23, 24, or 25 mg/m².

In one embodiment, a fixed dose of the anti-CD26 antibody is administered. In another embodiment, the fixed dose is determined based on the immunosuppressive agent administered to the subject.

The present invention is also directed to a method of inhibiting inflammation in a human patient having an idiopathic inflammatory myopathy (IIM), the method comprising administering to the patient an effective amount of an anti-CD26 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, wherein the patient’s CD3+ T cells and/or CD31+ endothelial cells express CD26.

The present invention is also directed to a method of treating an idiopathic inflammatory myopathy (IIM) in a human patient, the method comprising administering to the patient an effective amount of an anti-CD26 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, wherein the patient’s CD3+ T cells and/or CD31+ endothelial cells express CD26.

In one embodiment, the method is administered to a patient that has not received prior therapy (e.g., first line therapy). In another embodiment, the prior therapy is administration of an immunosuppressive agent.

In one embodiment, expression of CD26 is assayed by RT-PCR, in situ hybridization, RNase protection, RT-PCR-based assay, immunohistochemistry, enzyme linked immuosorbent assay, in vivo imaging, or flow cytometry. In another embodiment, the CD26 expression is assayed by immunohistochemistry.

The present invention is also directed to an antibody that specifically binds CD26 comprising the heavy chain and light chain nucleotide sequences comprising SEQ ID NO:15 and SEQ ID NO:16, respectively.

The present invention is also directed to an antibody that specifically binds CD26 for use in the treatment of a subject having an idiopathic inflammatory myopathy.

The present invention is also directed to the use of an antibody that specifically binds CD26 in the manufacture of a medicament for the treatment of an idiopathic inflammatory myopathy.

### BRIEF DESCRIPTION OF THE FIGURES

FIG. 1 shows percentage of CD26 expression as a function of fields of view (FoV) by immunohistochemical staining. (A) CD26 expression was quantified in IIM patients relative to healthy controls. (B) CD26 expression in IIM patients was further divided into patients having dermatomyositis (DM), polymyositis (PM), sporadic inclusion body myositis (IBM) and immune-mediated necrotizing myopathy (IMNM).

### DETAILED DESCRIPTION OF THE INVENTION

### 1. Definitions

In order that the present disclosure may be more readily understood, certain terms are first defined. As used in this application, except as otherwise expressly provided herein, each of the following terms shall have the meaning set forth below. Additional definitions are set forth throughout the application.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system. In one embodiment, an antibody is an intact antibody.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” includes monospecific, bispecific, multivalent or multi-specific, antibodies, as well as a single chain antibody.

As used herein, an “IgG antibody” has the structure of a naturally occurring IgG antibody, i.e., it has the same number of heavy and light chains and disulfide bonds as a naturally occurring IgG antibody of the same subclass. For example, an anti-CD26 IgG1, IgG2, IgG3 or IgG4 antibody consists of two heavy chains (HCs) and two light chains (LCs), wherein the two heavy chains and light chains are linked by the same number and location of disulfide bridges that occur in naturally occurring IgG1, IgG2, IgG3 and IgG4 antibodies, respectively (unless the antibody has been mutated to modify the disulfide bonds)

An “isolated antibody” refers to an antibody that is substantially free of other antibodies having different antigenic specificities (e.g., an isolated antibody that binds specifically to CD26 is substantially free of antibodies that bind specifically to antigens other than CD26). An isolated antibody that binds specifically to CD26 may, however, have cross-reactivity to other antigens, such as CD26 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-CD26 antibody binds specifically to CD26.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-CD26 antibody described herein, include:
(1) a Fab fragment (fragment from papain cleavage) or a similar monovalent fragment consisting of the VL, VH, LC and CH1 domains;
(2) a F(ab′)2 fragment (fragment from pepsin cleavage) or a similar bivalent fragment comprising two Fab fragments linked by a disulfide bridge at the hinge region;
(3) a Fd fragment consisting of the VH and CH1 domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

The term “CD26” refers to dipeptidyl peptidase 4 (DPP4). The terms CD26 and DPP4 are used interchangeably herein. The term “CD26” includes variants, isoforms, homologs, orthologs and paralogs. For example, antibodies specific for a human CD26 protein may, in certain cases, cross-react with a CD26 protein from a species other than human. In other embodiments, the antibodies specific for a human CD26 protein may be completely specific for the human CD26 protein and may not exhibit species or other types of cross-reactivity, or may cross-react with CD26 from certain other species, but not all other species (e.g., cross-react with monkey CD26 but not mouse CD26). The term “human CD26” refers to human sequence CD26, such as the complete amino acid sequence of human CD26 having GenBank Accession No. AH005372.3. The human CD26 sequence may differ from human CD26 of GenBank Accession No. AH005372.3 by having, e.g., conserved mutations or mutations in non-conserved regions and the CD26 has substantially the same biological function as the human CD26 of GenBank Accession No. AH005372.3.

A particular human CD26 sequence will generally be at least 90% identical in amino acid sequence to human CD26 of GenBank Accession No. AH005372.3 and contains amino acid residues that identify the amino acid sequence as being human when compared to CD26 amino acid sequences of other species (e.g., murine). In certain cases, a human CD26 can be at least 95%, or even at least 96%, 97%, 98%, or 99% identical in amino acid sequence to CD26 of GenBank Accession No. AH005372.3. In certain embodiments, a human CD26 sequence will display no more than 10 amino acid differences from the CD26 sequence of GenBank Accession No. AH005372.3. In certain embodiments, the human CD26 can display no more than 5, or even no more than 4, 3, 2, or 1 amino acid difference from the CD26 sequence of GenBank Accession No. AH005372.3.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity. An alignment for purposes of determining percent nucleic acid sequence identity can be carried out according to procedures known in the art, as described for example in EP 1 241 179 B1. For example, a skilled person can use publicly available computer software, such as using publicly available computer software such as BLAST, BLAST-2, ALIGN or Megalign (DNASTAR) software. A skilled person can determine appropriate parameters for measuring alignment, including any algorithms needed to achieve maximal alignment over the full length of the sequences subjected to comparison. According to a preferred embodiment, the % identity values can be generated using the WU-BLAST-2 computer program. According to a preferred embodiment, the following computer program and parameters are used: The identity values used herein are generated by the BLASTN module of WU-BLAST-2 set to the default parameters, with overlap span and overlap fraction set to 1 and 0.125, respectively. A % nucleic acid sequence identity value can be obtained by dividing (a) the number of matching identical nucleotides between a particular nucleic acid sequence as set forth herein which is subjected to comparison (e.g. a particular nucleic acid sequence characterized by a sequence identifier in the sequence listings), and the comparison nucleic acid molecule of interest to be compared, for example the number of matching identical nucleotides as determined by WL1-BLAST-2, by (b) the total number of nucleotide residues of the particular nucleic acid sequence as set forth herein which is subjected to comparison (e.g. a particular nucleic acid sequence characterized by a sequence identifier in the sequence listings).

A “patient” as used herein includes any patient who is afflicted with an IIM. The terms “subject” and “patient” are used interchangeably herein.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Diagnosis for IIM is generally based on the criteria of Targoff et al. (Targoff IN, Miller FW, Medsger Jr TA, Oddis CV. Classification Criteria for the Idiopathic Inflammatory Myopathies. Curr Opin Rheumatol, 1997; 9 (6):527-35) that include: symmetric proximal muscle weakness; elevations of serum levels of skeletal-muscle enzymes; electro-myographic features of muscle affection; infiltration, degeneration, or atrophy in muscle biopsies; presence of myositis-specific auto-antibodies; and/or typical skin rash of dermatomyositis.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system.

In one example, an “effective amount” is the amount of anti-CD26 antibody clinically proven to affect a significant decrease in the inflammatory response of an IIM. As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/m² dose, but rather as an absolute amount of the agent (e.g., the anti-CD26 antibody). For example, a 60 kg person and a 100 kg person would receive the same dose of the composition (e.g., 100 mg of an anti- CD26 antibody).

The term “body surface area based dose” as referred to herein means that a dose that is administered to a patient is calculated based on the surface area of the patient. For example, when a patient with 2.0 body surface area (BSA) requires 4 mg/m² of an anti-CD26 antibody, one can draw the appropriate amounts of the anti-CD26 antibody (i.e., 8 mg).

“Dosing interval,” as used herein, means the amount of time that elapses between doses of the anti-CD26 antibody disclosed herein being administered to a subject. Dosing interval can thus be indicated as ranges.

The term “dosing frequency” as used herein refers to the frequency of administering doses of the anti-CD26 antibody disclosed herein in a given time. Dosing frequency can be indicated as the number of doses per a given time, e.g., once a week or once in two weeks.

The terms “about once a week,” “once about every week,” “once about every two weeks,” or any other similar dosing interval terms as used herein means approximate number, and “about once a week” or “once about every week” can include every seven days ± two days, i.e., every five days to every nine days. The dosing frequency of “once a week” thus can be every five days, every six days, every seven days, every eight days, or every nine days. “Once about every two weeks” can include every fourteen days ± three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “CD26 positive” or “CD26 expression positive,” relating to CD26 expression, refers to the proportion of cells in a test tissue sample, typically comprising muscle cells, which the tissue sample is scored as expressing CD26

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate’s body of invading pathogens, cells or tissues infected with pathogens, cancerous or other abnormal cells, or, in cases of autoimmunity or pathological inflammation, normal human cells or tissues.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### 2. Methods of the Invention

One aspect of the invention relates to a method of inhibiting an immune response in a human patient having IIM comprising administering an effective amount of an anti-CD26 antibody to the patient. Another aspect of the invention relates to a method of treating IIM in a human patient, the method comprising administering to the patient an effective amount of an anti-CD26 antibody to the patient. In another aspect, the invention relates to a method of treating recurring IIM in a human patient comprising administering an effective amount of an anti-CD26 antibody to the patient. One aspect of the invention relates to a method of treating IIM in a human patient, the method comprising administering to the patient an effective amount of an anti-CD26 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5. In some embodiments, the methods of the invention further comprise administration of an immunosuppressive agent. In certain embodiments, the immunosuppressive agent is given before, concomitantly, or after the anti-CD26 antibody. When given concomitantly with the anti-CD26 antibody, the immunosuppressive agent can be given immediately prior to, simultaneously with, or immediately after administration of the anti-CD26 antibody. In certain embodiments, any of the present methods further comprise determining CD26 expression in a tissue sample.

In one embodiment, the invention includes a method of identifying a patient with an IIM who is likely to respond to an anti-CD26 antibody therapy, the method comprising: (a) determining the level of CD26 expression in a tissue sample; and (b) identifying the patient who is likely to respond to treatment if the tissue is a CD26 positive tissue sample. In one embodiment, the invention includes a method of selecting a patient with an IIM for anti-CD26 therapy, the method comprising: (a) determining the level of CD26 expression in a tissue sample; and (b) selecting the patient for anti-CD26 therapy if the tissue sample is CD26+. In certain embodiments, the tissue sample is a skeletal muscle tissue sample. In certain embodiments, the skeletal muscle sample comprises CD3+ T lymphocytes and/or CD31+ endothelial cells.

In one embodiment, the invention includes a method of treating an IIM in a human patient, comprising: administering to the patient an anti-CD26 therapy disclosed herein; wherein the patient is predicted to respond to treatment with the anti-CD26 antibody based upon CD26 expression in a tissue sample from the patient. In certain embodiments, the tissue sample comprises CD3+ T lymphocytes and CD31+ endothelial cells.

In some embodiments, the CD26 positive tissue sample comprises at least about 1%, at least about 2%, at least about 3%, at least about 4%, at least about 5%, at least about 7%, at least about 10%, at least about 15%, at least about 20%, at least about 25%, at least about 30%, at least about 40%, at least about 50%, at least about 60%, at least about 70%, at least about 80%, at least about 90%, or 100% cells expressing CD26. In certain embodiments, the tissue sample comprises CD3+ T lymphocytes and CD31+ endothelial cells.

In some embodiments, the identifying comprises determining CD26 expression in the tissue sample.

In some embodiments, CD26 expression is determined by receiving the results of an assay capable of determining CD26 expression.

### Measurement of CD26 Expression

In certain embodiments, identifying a patient suitable for a CD26 antibody therapy for the present methods includes measuring or assessing CD26 expression in a tissue sample comprising skeletal muscle. The methods of measuring or assessing the CD26 expression can be achieved by any methods applicable.

In order to assess CD26 expression, in one embodiment, a test tissue sample is obtained from the patient who is in need of the therapy. In some embodiments, the test tissue sample is a paraffin-embedded fixed tissue sample. In some embodiments, the test tissue sample is a formalin-fixed paraffin embedded (FFPE) tissue sample. In some embodiments, the test tissue sample is a fresh tissue (e.g., skeletal muscle) sample. In some embodiments, the test tissue sample is a frozen tissue sample. In some embodiments, the test tissue sample is a fresh frozen (FF) tissue (e.g., skeletal muscle) sample. In some embodiments, the test tissue sample is a cell isolated from a fluid. In some embodiments, the test tissue sample comprises CD3+ infiltrating T lymphocytes. In some embodiments, the test tissue sample comprises CD31+ endothelial cells. In some embodiment, the test tissue sample comprises CD3+ infiltrating T lymphocytes and CD31+ endothelial cells. In some embodiments, the test tissue sample is an archival tissue sample. In some embodiments, the test tissue sample is an archival tissue sample with known diagnosis, treatment, and/or outcome history. In some embodiments, the sample is a block of tissue. In some embodiments, the test tissue sample is dispersed cells. In some embodiments, the sample size is from about 1 cell to about 1 × 10⁶ cells or more. In some embodiments, the sample size is about 1 cell to about 1 × 10⁵ cells. In some embodiments, the sample size is about 1 cell to about 10,000 cells. In some embodiments, the sample size is about 1 cell to about 1,000 cells. In some embodiments, the sample size is about 1 cells to about 100 cells. In some embodiments, the sample size is about 1 cell to about 10 cells. In some embodiments, the sample size is a single cell.

In any of the methods comprising the measurement of CD26 expression in a test tissue sample, however, it should be understood that the step comprising the provision of a test tissue sample obtained from a patient is an optional step. That is, in certain embodiments the method includes this step, and in other embodiments, this step is not included in the method. It should also be understood that in certain embodiments the “measuring” or “assessing” step to identify, or determine the number or proportion of, cells in the test tissue sample that express CD26 is performed by a transformative method of assaying for CD26 expression, for example by performing a reverse transcriptase-polymerase chain reaction (RT-PCR) assay or an IHC assay. In certain other embodiments, no transformative step is involved and CD26 expression is assessed by, for example, reviewing a report of test results from a laboratory. In some embodiments, CD26 expression is assessed by reviewing the results of an immunohistochemistry assay from a laboratory. In certain embodiments, the steps of the methods up to, and including, assessing CD26 expression provides an intermediate result that may be provided to a physician or other healthcare provider for use in selecting a suitable candidate for the anti-CD26 antibody. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express CD26 is assessed by performing an assay to detect the presence of CD26 RNA. In further embodiments, the presence of CD26 RNA is detected by RT-PCR, in situ hybridization or RNase protection. In some embodiments, the presence of CD26 RNA is detected by an RT-PCR based assay. In some embodiments, scoring the RT-PCR based assay comprises assessing the level of CD26 RNA expression in the test tissue sample relative to a predetermined level.

In other embodiments, the proportion of cells that express CD26 is assessed by performing an assay to detect the presence of CD26 polypeptide. In further embodiments, the presence of CD26 polypeptide is detected by IHC, enzyme-linked immunosorbent assay (ELISA), in vivo imaging, or flow cytometry. In some embodiments, CD26 expression is assayed by IHC. In other embodiments of all of these methods, cell surface expression of CD26 is assayed using, e.g., IHC or in vivo imaging.

In other embodiments, the proportion of cells that express CD26 in the test tissue sample is assessed by flow cytometry. In some embodiments, the test tissue sample assayed by flow cytometry comprises CD3+ T lymphocytes and CD31+ endothelial cells. In some embodiments, the flow cytometry is a multiplex assay. In some embodiments, scoring the flow cytometry comprises detecting the expression of markers comprising CD26, CD3, CD31, and any combination thereof. In certain embodiments of any of the present methods, the proportion of cells that express CD26 in the test tissue sample is assessed by performing an assay to detect the presence of CD26 polypeptide. In some embodiments, the presence of CD26 polypeptide is detected by an immunohistochemistry assay. In some embodiments, the test tissue sample is a skeletal muscle biopsy. In some embodiments, the test tissue sample is a formalin-fixed paraffin embedded (FFPE) sample.

In some embodiments, the immunohistochemistry assay is a monoplex assay. In some embodiments, the immunohistochemistry assay is a multiplex assay. In some embodiments, the multiplex immunohistochemistry assay is capable of detecting the presence of CD26, CD3, CD31, or any combination thereof.

In some embodiments, the immunohistochemistry assay comprises contacting the tissue sample with the D6D8K rabbit anti-human CD26 (Cell Signaling Technology) (Abcam) monoclonal antibody. In some embodiments, the immunohistochemistry assay comprises contacting the skeletal muscle sample with an anti-CD26 antibody comprising heavy and light chain variable regions comprising the sequences set forth in SEQ ID NOs: 3 and 5, respectively.

In some embodiments, the immunohistochemistry assay is scored at a low magnification. In some embodiments, low magnification is about 20X. In some embodiments, the immunohistochemistry assay is scored at high magnification. In some embodiments, high magnification is about 40X.

In some embodiments, the immunohistochemistry assay is scored by an image analysis software. In some embodiments, the immunohistochemistry assay is scored by pathologist visual immune score. In some embodiments, the immunohistochemistry assay is scored manually.

In some embodiments, scoring the immunohistochemistry assay comprises assessing the proportion of cells in the test tissue sample that express CD26. In some embodiments, scoring the immunohistochemistry assay comprises assessing the proportion of immune cells in the test tissue sample that express CD26. In some embodiments, scoring the immunohistochemistry assay comprises assessing the proportion of CD3+ T cells in the test tissue sample that express CD26. In some embodiments, scoring the immunohistochemistry assay comprises assessing the proportion of CD31+ endothelial cells in the test tissue sample that express CD26.

### 3. CD26 Antibodies

In one aspect, the invention features methods of using an anti-CD26 antibody in the treatment of an IIM. Anti-human-CD26 antibodies (or VH/VL domains derived therefrom) suitable for use in the invention can be generated using methods well known in the art. Alternatively, art recognized anti-CD26 antibodies can be used.

In some embodiments, the anti-CD26 antibody is produced from the hybridoma cell line deposited on Sep. 11, 2012 under the Budapest Treaty at the Centro di Biotecnologie Avanzate (CBA)--Interlab Cell Line Collection (ICLC) of Genoa (L. go R. Benzi, 10, Genoa, Italy) as deposit PD 12002. In another embodiment, the anti-CD26 antibody used in the methods of the invention bind the same epitope as an antibody produced by the hybridoma cell line deposited at CBA-ICLC of Genoa (Italy) deposited as PD 12002.

In some embodiments, the anti-CD26 antibody is begelomab comprising heavy and light chains comprising the sequences shown in SEQ ID NOs:1 and 2, respectively, or antigen binding fragments and variants thereof, as described in U.S. Pat. Nos. 9,376,498 and 10,208,126, the teachings of which are hereby incorporated by reference.

In other embodiments, the antibody has the heavy and light chain CDRs or variable regions of begelomab. Accordingly, in one embodiment, the antibody comprises CDR1, CDR2, and CDR3 domains of the VH region of begelomab having the sequence set forth in SEQ ID NO:3, and CDR1, CDR2 and CDR3 domains of the VL region of begelomab having the sequence set forth in SEQ ID NO:5. In another embodiment, the antibody comprises CDR1, CDR2 and CDR3 domains comprising the sequences set forth in SEQ ID NOs:7, 8, and 9, respectively, and CDR1, CDR2 and CDR3 domains comprising the sequences set forth in SEQ ID NOs:10, 11, and 12, respectively. In another embodiment, the antibody comprises VH and/or VL regions comprising the amino acid sequences set forth in SEQ ID NO:3 and/or SEQ ID NO: 5, respectively. In another embodiment, the antibody comprises heavy chain variable (VH) and/or light chain variable (VL) regions encoded by the nucleic acid sequences set forth in SEQ ID NO:4 and/or SEQ ID NO:6, respectively. In another embodiment, the antibody competes for binding with and/or binds to the same epitope on CD26 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:3 or SEQ ID NO:5).

In some embodiments, the anti-CD26 antibody or antigen-binding portion thereof cross-competes with begelomab for binding to human CD26. In other embodiments, the anti-CD26 antibody or antigen-binding portion thereof binds to the same epitope as begelomab.

In some embodiments, the anti-CD26 antibody is codon-optimized for expression in a host cell. In one embodiment, the anti-CD26 antibody is begolomab that is codon optimized for expression in Chinese Hamster Ovary (CHO) cells. In another embodiment, the codon optimized begelomab comprises the heavy chain and light chain of SEQ ID NOs: 15 and 16, respectively.

In one embodiment, the anti-CD26 antibody is an antibody or antigen-binding fragment thereof described in U.S. Pat. No. 7,658,923 (for example, 1F7); U.S. Pat. No. 7,462,698 (for example, CM03), U.S. Pat. No. 8,771,688, and EP Pat. No. 3 348 276 A1. Antibodies or antigen-binding fragments thereof that compete with any of the above-referenced art-recognized antibodies for binding to CD26 also can be used.

In certain embodiments, an anti-CD26 antibody is used to determine CD26 expression. In some embodiments, an anti-CD26 antibody is selected for its ability to bind to CD26 in formalin-fixed, paraffin-embedded (FFPE) tissue specimens. In other embodiments, an anti-CD26 antibody is capable of binding to CD26 in frozen tissues. In further embodiments, an anti-CD26 antibody is capable of distinguishing membrane bound, cytoplasmic, and/or soluble forms of CD26.

### 4. Immunosuppressive Agents

In some embodiments, the methods of the invention feature using one or more immunosuppressive agents in combination with the anti-CD26 antibody to treat an IIM. In one embodiment, the immunosupressive agent is considered the standard of care for treatment of the IIM. An “immunosupressive agent” is a compound that inhibits or prevents the activity of the immune system. In one embodiment, the immunosuppressive agent is a corticosteroid. Corticosteroids are well known in the art and can comprise in particular mineralocorticoids and glucocorticoids. Glucocorticoids can be antiinflammatory agents. As used herein, the term corticosteroids can include steroids which can be in particular produced in the adrenal cortex of vertebrates, as well as can encompass synthetic corticosteroids or synthetic or natural corticosteroid analogs, including compounds that mimic the activity of natural steroid hormones, such as e.g. cortisone and hydrocortisone. Corticosteroid analogs may in particular encompass synthetic or natural chemical compounds which resemble in structure and/or function any of naturally occurring steroids elaborated by the adrenal cortex.

One or more corticosteroids can be selected from the group consisting of alclometasone dipropionate, amcinonide, amcinafel, amcinafide, beclamethasone, betamethasone, betamethasone dipropionate, betamethasone valerate, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone, diflorasone diacetate, dichlorisone, esters of betamethasone, fluazacort, flucetonide, flucloronide, fludrotisone, fluorocortisone, flumethasone, flunisolide, fluocinonide, fluocinolone, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, loteprendol, medrysone, meprednisone, methylprednisone, methylprednisolone, 6-methylprednisolone, mometasone furoate, paramethasone, paramethasone acetate, prednisone, prednisolone, prednidone, prednicarbate, triamcinolone acetonide, triamcinolone hexacatonide, tixocortol prednisolone, and triamcinolone, pharmaceutically acceptable salts thereof, derivatives thereof, and mixtures thereof. In other embodiments, the immunosuppressive agents include methotrexate, azathioprine, mycophenolate mofetil, alkylating agents such as cyclophosphamide, nitrosoureas, and platinum compounds, and monoclonal antibodies such as rituximab, tocilizumab, alemtuzumab, and saflimumab. In one embodiment, immunosuppressive agents include pharmaceutically acceptable salts, acids or derivatives of any of the above; as well as combinations of two or more of the above.

### 5. Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-CD26 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-CD26 antibody is administered intravenously.

### 6. Patient Populations

Provided herein are clinical methods for treating an IIM in human patients using an anti-CD26 antibody. In some embodiments, the patients are further administered one or more immunosuppressive agents.

In one aspect, the present invention is directed to a method for treating an idiopathic inflammatory myopathy (IIM) in a subject in need thereof. A therapeutic regimen results in better therapeutic outcomes (e.g., objective response rate and disease control rate) in a patient population with CD26 positive lymphocytic and/or endothelial cells than in a general patient population having a mix of CD26-negative CD26-positive cells. In order to improve the treatment of IIM, in one aspect, the present invention provides identifying a patient as having a CD26-positive cells and providing a therapy of an anti-CD26 antibody.

IIMs are rare conditions, they include dermatomyositis (DM), polymyositis (PM), necrotizing myopathy (NM) and inclusion body myositis (IBM) (Carstens et al, Clin. Exp. Immunol. 175:425-438 (2014)), each of which have unique clinical presentations.

Dermatomyositis (DM) classically presents with proximal symmetric muscle weakness and associated characteristic rashes of the skin such as Gottron papules on the dorsal sides of the hands and fingers, a periorbital oedema, and erythema of the face (heliotrope rash), the anterior upper chest (V-sign) or the posterior neck (shawl sign). The muscle inflammation causes proximal weakness which can develop acutely (within several days) or subacutely (within several weeks up to a few months). The patients suffer from impaired walking and climbing stairs as well as lifting their arms and heavy objects (Schmidt, J. Neuromuscular Dis. 5:109-129 (2018)). Several variants of classical DM exist such as: clinically amyopathic DM (CADM) (Sun Y et al, Rheumatol. Int. 33:1295-1302 (2013)), adermatopathic DM (“dermatomyositis sine dermatitis”) (Iaccarino et al, J. Autoimmun. 48-49:122-127 (2014)), juvenile dermatomyositis (JDM).

Histopathologic signs of DM consist of a perimysial inflammation, perifascicular atrophy and perifascicular elevation of MHC class I, binding of complement to capillaries and the surface of the sarcolemma and reduction of capillaries. The typical pathological feature of perifascicular atrophy is more common in JDM than in DM (Dalakas, New Engl. J. Med. 372:1734-1747 (2015)).

Polymyositis (PM) is a disease of adults over the age of 20 years and is more common in women (Dimachkie et al, Semin. Neurol. 32:227-236 (2012)). Myalgias and tenderness are common, but not presenting complaints. Dysphagia occurs in one-third of patients, and mild facial weakness is occasionally present. Rash or other signs of inflammation of the skin do not occur in PM. The diagnosis of PM should be made by exclusion of all other types. Serum CK levels may be elevated up to 50 times the upper limit of normal in the subacute active phase (Mandel et al, Int. J. Mol. Sci. 18:1084-1094 (2017)). Histopathologic hallmarks of PM include an “invasion” of muscle fibers by presence of endomysial cytotoxic CD8+ T-cells and widespread upregulation of MHC class I. (Hoogendijk et al, Neuromuscular Dis. 14:337-345 (2004)).

Necrotizing myopathy (NM) leads to an acute or subacute proximal weakness of the arms and legs. The disease course is often more rapid and more severe compared to DM and PM. The muscle enzymes are usually very high with a 20-50 fold elevated CK. Two auto-antibodies have been shown to be associated with NM. Anti-signal recognition particle (SRP) antibodies are used as serological markers of necrotizing myopathy, which is characterized by many necrotic and regenerative muscle fibers without or with minimal inflammatory cell infiltration (Suzuki et al, Orphanet J. Rare Dis. 10:61-70 (2015)). Anti-SRP antibodies are expected in about 10 to 20% of patients with NM, although the rate of detection ranges largely (from 0% to 54%). Anti-SRP can be associated with a cardiomyopathy and a severe disease course with muscular atrophy, interstitial lung disease (ILD) and dysphagia. The second autoantibody that was identified in up to 60% of certain NM cohorts is anti-HMGCR (Christopher-Stine et al, 2010). An association with malignancy has been shown to be higher in patients with necrotizing autoimmune myopathies without myositis-specific antibody or with anti-HMGCR antibodies, older than 50 years, within 3 years of diagnosis, as such patients have an increased risk of cancer and a poorer prognosis (Allenbach et al, Brain 139:2131-2135 (2016)). Conversely, dermatomyositis can no longer be considered as the main idiopathic inflammatory myopathy with an increased risk of cancer.

The histological picture in NM displays scattered necrotic myofibers of varying degree, moderate and mostly focal upregulation of MHC class I, particularly in areas with necrotic fibers, and binding of complement to the sarcolemma (Allenbach et al, Neuropath. Appl. Neurobiol. 43:62-81 (2017)). Some inflammatory T-cells and other immune cells may be present around these focal spots, but there are no primary inflammatory lesions. Necrotic fibers typically display a secondary invasion by macrophages for clearance of the cell debris.

Sporadic inclusion body myositis (IBM) is the most common acquired muscle disease presenting in people over 50 years of age, this chronic, debilitating condition affects more men than women (3:1 ratio) (Price et al, J. Neuromuscular Dis. 3:67-75 (2016)). Clinically, it is characterized by slowly progressive weakness and muscle wasting predominantly of the quadriceps and long finger flexor muscles (Rothwell et al, Curr. Opin. Rheumatol. 29-639-644 (2017)). The typical pattern of muscle involvement includes weakness of the long finger flexors, the quadriceps, the tibialis anterior and, usually to a lesser extent, all other muscles of arms and legs. The progression is much slower than that of other forms of myositis, but continues relentlessly and leads to a profound muscle atrophy. As the disease progresses, patients continue to lose muscle strength at a rate of 3.5-16.8% per year several years after disease onset, most patients require assistance with daily activities, as well as needing ambulatory aids such as a cane, walker or wheelchair (Price et al, J. Neuromuscular Dis. 3:67-75 (2016)). Weakening or dysfunction of the pharyngeal and esophageal muscles can lead to dysphagia (difficulty with swallowing), which is experienced by 40-80% of patients during the course of IBM, and can result in choking, weight loss, aspiration and pneumonia. The weakness often leads to injurious falls and the dysphagia may cause aspiration pneumonia, which explains a higher rate of mortality in these patients. There is evidence that the pathogenesis of IBM is multifactorial, including inflammatory and degenerative changes and mitochondrial abnormalities.

In IBM, inflammatory features in muscle biopsy specimens suggest an immune-mediated component to disease pathogenesis. In addition, circulating anti-Ro autoantibodies may be found in around 20% of patients and recent work has identified cytosolic 50-nucleotidase 1A (anticN-1A) autoantibodies in around one-third of patients. Mitochondrial DNA deletions in COX-deficient muscle fibres correlate with T-lymphocyte infiltration and muscle fibre atrophy, suggesting a mechanistic link between these inflammatory and degenerative disease processes (Rothwell et al, Curr. Opin. Rheumatol. 29:639-644 (2017)).

Overlap Myositis (overlap syndrome with myositis, OM) is defined by the association of myositis with overlap connective tissue disease features, such as Raynaud’s phenomenon, arthritis, Mechanic’s hands, Trigeminal neuropathy and interstitial lung disease (ILD), as well as features of systemic sclerosis (SSc) and lupus, which most commonly are present at the time of myositis diagnosis (Senecal et al, Arthritis & Rheumatol. 69:878-884 (2017)). A high elevation of muscle enzymes including CK is usually present in OM (10-50 fold). Furthermore, the concept of OM has been supported by the identification of 15 specific autoantibodies and recognition of their striking association with distinct clinical phenotypes. Cytoplasmic autoantibodies are Jo-1 and non-Jo-1 synthetases and MDA-5 (CADM140) while Nuclear autoantibodies are U1 RNP, U3 RNP (fibrillarin), U5 RNP, U11-12 RNP, PM-Scl, Ku, nup (nucleoporins), CENP-B, Th/To, RuvB-like ½, DNA topoisomerase I, RNA polymerase III. The most common is the Jo-1 antibody, which is observed in 30% myositis patients (Lega et al, Autoimmunity Rev. 13:883-891 (2014)) while anti-PM/Scl, which is commonly associated with systemic sclerosis, present in up to 12% of myositis cases. Anti-UsnRNP antibodies are observed in 3-8% of myositis patients and associated with mixed connective tissue disease (CTD). Whereas patients with anti-U-snRNP often have a good prognosis, anti-PM/Scl may indicate a more severe course and insufficient treatment response. Anti-Ku antibodies are associated with systemic sclerosis, systemic lupus erythematosus (SLE) and other mixed and undifferentiated CTD: in overlap syndromes with myositis, they are present in up to 19% of the case (Lega et al, 2014).

In one embodiment, the method comprises treatment of a patient that has been diagnosed with an IIM. One of the clinical sign is the development of symmetrical muscle weakness and muscle fatigue (Lundberg et al, 2016), most prominent in proximal muscles, and signs such as laboratory investigations supporting skeletal muscle inflammation and muscle fibre degeneration and repair (regeneration). The most easily available test to demonstrate skeletal muscle involvement is elevated serum levels of muscle enzymes, the most often tested is creatine phosphokinase (CK), others are lactate dehydrogenase (LD), aspartate transaminase (AST) and alanine transaminase (ALT), and less commonly used is aldolase. Importantly, elevated serum levels of muscle enzyme are not specific for myositis as elevated levels could be seen in many other myopathies and normal muscle enzymes do not exclude myositis.

Muscle biopsy with histopathological evaluation is a very useful tool and constitutes the core in the diagnostic work-up of adult IIMs both to confirm skeletal muscle inflammation and to exclude other myopathies. A muscle biopsy is recommended to classify IIM patients without a typical dermatomyositis skin rash into IBM, INM. Immunohistochemistry staining such as for major histocompatibility complex (MHC) class I, for T cells and macrophages may be helpful to confirm signs of inflammation.

Electromyography (EMG) is another tool to detect myopathies and some changes may also distinguish between necrosis and denervation, such as the size, shape, and recruitment pattern of the motor units potential, although there are no specific EMG findings for myositis.

Myositis-associate autoantibodies (MAAs) and myositis-specific autoantibodies (MSAs) have been extensively demonstrated to correlate with specific clinical manifestations since they are important biomarkers for myositis aiding in diagnosis and helping to classify patients into more homogeneous group (Betteridge et al, 2016). Myositis autoantibodies may therefore aid in predicting additional clinical complications and response to treatment.

MAAs and MSAs are detected by a variety of methods: one of the most commonly used is immunofluorescence but the results are often negative or non -specific. The gel precipitation assay has also been used but, due to the low sensitivity, cannot detect all myositis autoantibodies. These assays have been superseded by ELISAs using either a generic or a specific antigen source. However, the binding of the antigen to a plastic plate can result in the loss of some epitopes and currently only a limited number of MAAs/MSAs can been detected. Radiolabelled immunoprecipitation (IPP) has been regarded as the gold standard testing method for autoantibody serology due its high sensitivity and ability to detect a wide repertoire of known and unknown autoantigen targets.

### 7. Treatment Protocols

In one aspect, suitable treatment protocols for treating an IIM in a human patient include administering to the patient an effective amount of an anti-CD26 antibody. In another embodiment, the treatment further requires administration of one or more immunosuppressive agents.

In some embodiments, a suitable treatment protocol for treating an IIM in a human patient include, for example, administering to the patient an effective amount of each of:

(a) an anti-CD26 antibody, such as one comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5,

wherein the method comprises at least one administration cycle, wherein the cycle is a period of 1 week, wherein for each of the at least one cycles, at least 5 doses of the anti-CD26 antibody are administered at a dose of about 4, 5, 6, 7, 8, 9, 10, 11, 12, 13, 14, 15, 16, 17, 18, 19, 20, 21, 22, 23, 24, or 25 mg/m². In one embodiment, the cycle is a period of 4 weeks, wherein for each of the at least one cycles, at least 5 doses of the anti-CD26 antibody are administered at a dose of about 4, 5, 6, 7, 8, 9, 10, 11, 12, 13, 14, 15, 16, 17, 18, 19, 20, 21, 22, 23, 24, or 25 mg/m² is administered one daily for five days followed by 3 administrations per week for 3 weeks. In one embodiment, the cycle is a period of 5 weeks, wherein for each of the at least one cycles, at least 5 doses of the anti-CD26 antibody are administered at a dose of about 4, 5, 6, 7, 8, 9, 10, 11, 12, 13, 14, 15, 16, 17, 18, 19, 20, 21, 22, 23, 24, or 25 mg/m² is administered one daily for five days followed by 3 administrations per week for 4 weeks. In one embodiment, the at least 5 doses in the first week are administered on consecutive days.

Body surface area (BSA) can be calculated according to any known method. For example, the body surface area (BSA) of a patient can be calculated according to the Mosteller formula of BSA (m²)=([Height (cm)xWeight (kg)]/3600)^{½} (Mosteller R D., N Engl J Med 1987 Oct. 22; 317(17):1098, which is incorporated herewith by reference) or according to the DuBois and DuBois formula of BSA (m²)=0.20247×Height (m)^{0.725}×Weight (kg)^{0.425} (DuBois D; DuBois E F., Arch Int Med 1916 17:863-71, which is incorporated herewith by reference). In one embodiment, the Mosteller formula is used for calculating the body surface area (BSA) of a patient.

In certain embodiments, the anti-CD26 antibody is administered at a dose of about 4 mg/m² to 25 mg/m² daily for 1, 2, 3, 4, or 5 weeks.

In a further embodiment, one or more immunosuppressive agents are administered. In one embodiment, at least one immunosuppressive agent is administered intravenously. In one embodiment, at least one immunosuppressive agent is administered orally. In one embodiment, at least one immunosuppressive agent is administered topically.

In one embodiment, the one or more immunosuppressive agents are administered using a body surface area-based dosing.

### 8. Kits and Unit Dosage Forms

Also within the scope of the present invention are diagnostic kits comprising an anti-CD26 antibody for assaying CD26 expression as a biomarker for screening patients for the immunotherapy or for predicting the efficacy of the immunotherapy. Kits typically include a label indicating the intended use of the contents of the kit and instructions for use. The term “label” includes any writing, or recorded material supplied on or with the kit, or which otherwise accompanies the kit. In certain embodiments of a diagnostic kit, a first anti-CD26 antibody for assaying, detecting, and/or quantifying CD26 expression is co-packaged with at least one therapeutic antibody (e.g., a second anti-CD26 antibody). In some embodiments, the kit further comprises an immunosuppressive agent to be co-administered with the therapeutic anti-CD26 antibody.

In certain embodiments, the diagnostic kit comprises an anti-human CD26 monoclonal antibody for assaying, detecting, and/or quantifying CD26 expression. In one embodiment, the diagnostic kit comprises the rabbit monoclonal antibody D6D8K (Abcam).

Also provided herein are therapeutic kits which include a pharmaceutical composition containing an anti-CD26 antibody, such as begelomab, in a therapeutically effective amount adapted for use in the preceding methods. In certain embodiments of a therapeutic kit, the anti-CD26 antibody is co-packaged with an immunosuppressive agent for co-administration. The kits optionally also can include instructions, e.g., comprising administration schedules, to allow a practitioner (e.g., a physician, nurse, or patient) to administer the composition contained therein to a patient having an IIM. The kit also can include a syringe.

Optionally, the diagnostic and/or therapeutic kits include multiple packages of the single-dose pharmaceutical compositions each containing an effective amount of the anti-CD26 antibody for a single administration in accordance with the methods provided above. Instruments or devices necessary for administering the pharmaceutical composition(s) also may be included in the kits.

In one embodiment, the present invention provides a kit for treating a patient afflicted with an IIM, the kit, for example, comprising:
(a) a dose of an anti-CD26 antibody, such as one comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5;
(b) optionally one or more immunosuppressive agents; and
(d) instructions for using the anti-CD26 antibody, and one or more immunosuppressive agents in the methods described herein.

The present invention is further illustrated by the following examples which should not be construed as further limiting. The contents of all references cited throughout this application are expressly incorporated herein by reference.

### EXAMPLES

### EXAMPLE 1 CD26 Expression in Skeletal Muscle of IIM Patients

Expression of CD26 was analyzed in inflamed skeletal muscle of patients affected by IIM and to what extent and the localization of CD26 reflect clinical, serological and histological disease characteristics.

Consecutive individuals (n=37) who underwent muscle biopsy were included in the study. Thirty-two patients were classified as having DM, PM, IBM or IMNM while five participants had no evidence of muscle inflammation of other diseases and served as controls. Clinical features (presence and degree of muscle weakness, myalgias, interstitial lung disease, ILD, dysphonia, dysphagia, arthralgias, arthritis, myocarditis and DM rash, defined as the presence of heliotrope rash or Gottron’s sign) and serum muscle enzymes levels (creatine kinase, CK, and aldolase) of patients were documented at the time of biopsy.

The Medical Research Council (MRC) scale was used by the examining physician to serially quantify muscle strength and calculate the manual muscle test 8 (MMT-8) score at each visit. For analysis purposes, MRC scale was converted to Kendall’s 0-10 scale. Right and left-side strength assessments of deltoids, biceps brachii muscles, wrist extensors, hip flexors and extensors, quadriceps and ankle dorsi flexors were merged and the average was used for computations.

Perifascicular atrophy, presence and localization of necrosis and regeneration, and characteristics of inflammatory infiltrates were defined by histochemistry and immunofluorescence on all biopsies. Frozen sections of all muscle biopsies were obtained by cutting 7 µm-thick slices. CD26 expression was evaluated using the monoclonal antibodies (mAb) clone D6D8K (Abcam, dilution 1:50) and begelomab (2 mg/ml) after acetone fixation. Antigen expression was determined by immunofluorescence using antimouse AlexaFluor488 IgG2b secondary antibody as second-step reagent. Placental tissues, which express large amounts of the antigen, were used as positive controls. Isotype-matched control antibody was employed in parallel to verify the specificity of the staining. Nuclei were revealed by 4′,6-diamidino-2-phenylindole (DAPI), and anti-laminin (LSBio, dilution 1:200) followed by anti-chicken AlexaFluor633 secondary antibody was employed to identify muscle fibers. Images were captured using Ultraview Perkin Elmer laser scanning confocal microscope and analyzed. CD26 expression was quantified using the Java-based image processing program ImageJ, which calculates CD26 staining as % of field of view (FoV). At least four representative images were collected for each of the 37 participants and analyzed. The mean of the percentages obtained for each muscle biopsy was then used for statistical analysis. Eight biopsies out of 37 (four DM, two PM, one IBM and one IMNM) were further analyzed by immunofluorescence for the simultaneous expression of CD3 and CD31 antigens to identify T lymphocytes and endothelial cells, respectively. The rabbit anti-human CD31 mAb (LSBio, dilution 1:100) and the rabbit anti-human CD3 antibody (Abcam, dilution 1:50) were used for this purpose, and the goat anti-rabbit AlexaFluor546-conjugated mAb and the goat PE-conjugated anti-rabbit mAb were respectively employed as second-step reagents.

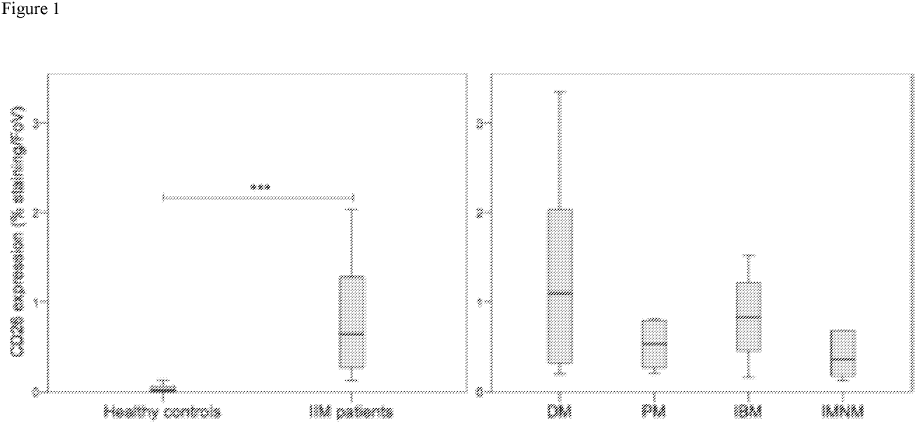

Categorical variables were expressed as percentages and absolute frequencies, while continuous variables as medians [IQR]. Two-tailed Mann-Whitney’s U-test was used to compare the level of muscular CD26 expression among patients with or without each clinical and histologic feature. Kruskal-Wallis nonparametric ANOVA was employed for the same comparison analysis among patients with IIM with different myositis subtypes. Correlations between CD26 expression and continuous variables including mean muscle test 8 (MMT-8) score, the level of muscle weakness in each muscle group and CK and aldolase serum levels were performed using two-tailed Spearman’s Rank correlation coefficient. For some analyses, patients were subdivided into two groups based on whether the percentage of CD26-positive field of views (FoV) at muscle biopsy was higher or lower than 0.645, the median value among all patients in study. Specifically, patients with a value ≥0.645 were defined as having a higher or more prominent CD26 expression, while those with a value <0.645 were considered as having reduced levels of CD26 in skeletal muscle. (FIG. 1.) For comparisons of categorical and continuous variables between these patient groups we employed Chi-square test or Fisher’s exact test, as appropriate, and Mann-Whitney’s U-test, respectively. The contribution of categorical or continuous variables in predicting study outcomes was determined using generalized linear models with gamma distribution of the dependent variables and log function as a link function for multivariate analysis. Poisson log-linear regression was used to define whether CD26 expression predicts the number of steroid-sparing immunosuppressive agents attempted before achieving muscle disease stabilization for at least six months or improvement based on MMT-8 and CK levels. Data were analyzed using Microsoft Excel® 2013 and IBM SPSS® version 21. A 2-sided p value ≤0.05 was considered statistically significant.

Ten (31%) patients with DM, twelve (37.5%) with PM, four (12.5%) with IBM and six (19%) with IMNM and 5 healthy controls were considered.

Immunofluorescence studies revealed that CD26 is expressed in all biopsies from patients with IIM, albeit with varying degrees of expression and different localization. No signal was ever detected in muscle biopsies of healthy controls. The median [IQR] CD26 expression (% staining/FoV) was 0.645 [0.251-1.302] for patients with IIM vs. 0.02 [0.0045-0.095] for healthy controls (p<0.001).

CD26 was consistently expressed in muscle tissue of all 32 analyzed patients with IIM by infiltrating and stromal cells.

Patients with DM express high levels of CD26. No difference in CD26 expression was found between females and males and any correlation between the extent of CD26 expression and age at onset or disease duration was observed.

The extent of CD26 expression was significantly higher in tissues characterized by myofiber necrosis than in those where this histological feature was not detected, while CK or aldolase levels did not correlate with the degree of CD26 expression.

The extent of CD26 expression was significantly higher in tissues characterized by myofiber necrosis than in those where this histological feature was not detected, while CK or aldolase levels did not correlate with the degree of CD26 expression.

Besides DM diagnosis and the presence of necrosis, vascular inflammation is associated with CD26 expression. CD26 was significantly more expressed in tissues of patients with perimysial CD3+ T lymphocytes than in those without and in tissues of patients presenting perivascular CD68+ macrophages and CD3+ T cells compared with those without these inflammatory infiltrates.

Two main cell populations were found to preferentially express CD26, i.e. CD3+ T lymphocytes and CD31+ endothelial cells. Not all infiltrating T cells express CD26, making it unlikely that its apparent more prominent expression in tissues of patients with DM is associated with the extent of perivascular infiltration. CD31+ endothelial cells represent a second major source of CD26 and in particular in patients with DM virtually all endothelial cells expressed the molecule. This feature appears to involve the overall microvasculature of the skeletal muscle, regardless of the presence of vascular injury or of perivascular infiltrating inflammatory cells. Although detectable in some specific areas, the expression of CD26 by endothelial cells was not a universal characteristic of the skeletal muscle of patients with PM, IBM and IMNM, possibly reflecting the less extensive vascular involvement in the pathogenesis of the muscular inflammation characteristic of the latter conditions.

The overall muscle strength at the time of biopsy was not influenced by the degree of CD26 expression. In contrast, MMT-8 at last visit was lower in patients with higher CD26 expression compared with those with reduced muscular levels of CD26 at diagnosis. Patients with higher CD26 expression at diagnosis developed at later times decreased strength in several muscle groups, including biceps, wrist extensors, hip flexors and quadriceps. Mean strength in these muscles as well as strength in neck flexors at last visit decreased with increasing percentages of CD26-positive FoV at diagnosis (all p≤0.02). Analyzed parameters are summarized in Table 1.

TABLE 1

Disease activity in patients with different CD26/DPP4 expression levels

| | High CD26 | Reduced CD26 |
|---|---|---|
| Mean MMT-8 | 138.2 [129-144.9] | 145.7 [136-149.2] |
| MMT-8 at last visit | 140.5 [129-145.2]* | 149 [139-150] |
| Mean neck flexors strength | 9.1 [8.1-10] | 10 [9.3-10] |
| Neck flexors at last visit | 10 [9-10] | 10 [10-10] |
| Mean arm abductors strength | 8.9 [6.9-9.7] | 9.5 [8.4-10] |
| Arm abductors strength at last visit | 9.5 [7.9-10] | 10 [8.5-10] |
| Mean biceps strength | 8.8 [8-9.6]** | 10 [9.4-10] |
| Biceps strength at last visit | 9.5 [8.3-10] | 10 [10-10] |
| Mean wrist extensors strength | 9.5 [7.5-9.8]* | 10 [9.5-10] |
| Wrist extensors strength at last visit | 9.2 [7-10] | 10 [10-10] |
| Mean hip flexors strength | 6 [5-8.2]** | 8.7 [8-9.2] |
| Hip flexors strength at last visit | 6 [4.5-8.5]** | 9 [8-10] |
| Mean quadriceps strength | 9.4 [7.7-9.7]** | 10 [9.9-10] |
| Quadriceps strength at last visit | 8.7 [7.8-10]* | 10 [10-10] |

*p<0.05, **p<0.01
Variables were expressed as medians [IQR]. Bivariate comparisons were made using Mann-Whitney’s U-test.
MMT-8 - manual muscle test 8

### Example 2 Treatment of IIM Patients With Anti-CD26 Antibody

One IIM-confirmed patient was treated with begelomab, murine monoclonal antibody against CD26, (2 mg/ml) concentrate for solution by infusion. Begelomab was intravenously administered to the patient at the dosage of 4 mg/m² daily for 5 consecutive days. This therapeutic choice was driven by the observation of the extensive expression of the T lymphocyte activation antigen CD26 in the patient’s muscle and skin tissues. At the end of the 5-day cycle of begelomab, skin lesions almost completely healed, with the disappearance of the erythematous rashes and ulcers, the bloody diarrhea ceased and CRP levels normalized.

Following DM flare, a new 5-day cycle of begelomab (4 mg/m² daily) followed by maintenance therapy every other day for additional eleven infusions (one infusion of 4 mg/m² daily) was administrated. The treatment accomplished a considerable clinical response few days after begelomab initiation. Gastrointestinal symptoms resolved completely and skin ulcers healed. Although severe muscle weakness persisted, improvement in dysphagia allowed the patient to gradually start semi-solid oral feeding. In the follow-up period, no DM exacerbation occurred and the patient’s quality of life improved dramatically.

### SEQUENCES

SEQ ID NO:1 Heavy Chain Constant Region Amino Acid Sequence; Anti-CD26 mAb (begelomab)

AKTTPPSVYPLAPGCGDTTGSSVTLGCLVKGYFPESVTVTWNSGSLSSSV
HTFPALLQSGLYTMSSSVTVPSSTWPSQTVTCSVAHPASSTTVDKKLEPS
GPISTINPCPPCKECHKCPAPNLEGGPSVFIFPPNIKDVLMISLTPKVTC
VVVDVSEDDPDVQISWFVNNVEVHTAQTQTHREDYNSTIRVVSTLPIQHQ
DWMSGKEFKCKVNNKDLPSPIERTISKIKGLVRAPQVYILPPPAEQLSRK
DVSLTCLVVGFNPGDISVEWTSNGHTEENYKDTAPVLDSDGSYFIYSKLN
MKTSKWEKTDSFSCNVRHEGLKNYYLKKTISRSPGK

SEQ ID NO:2 Light Chain Constant Region Amino Acid Sequence; Anti-CD26 mAb (begelomab)

RADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIVK
SFNRNEC

SEQ ID NO:3 Heavy Chain Variable Region (VH) Amino Acid Sequence; Anti-CD26 mAb (begelomab)

QVQLQQSGAELVKPGASVKLSCKASGYTFRSYDINWVRQRPEQGLEWIGW
IFPGDGSTKYNEKFKGKATLTTDKSSSTAYMQLSRLTSEDSAVYFCARWT
VVGPGYFDVWGAGTTVTVSS

SEQ ID NO:4 Heavy Chain Variable Region (VH) Nucleotide Sequence; Anti-CD26 mAb (begelomab)

caggtccagctgcagcagtctggagctgaactggtaaagcctggggcttc
agtgaagttgtcctgcaaggcttctggctacaccttcagaagttatgata
taaactgggtgagacagaggcctgaacagggacttgagtggattggatgg
atttttcctggagatggtagtactaagtacaatgagaagttcaagggcaa
ggccacactgactacagacaaatcctccagcacagcctacatgcagctca
gcaggctgacatctgaggactctgctgtctatttctgtgcaagatggacg
gtagtaggcccagggtacttcgatgtctggggcgcagggaccacggtcac
cgtctcctca

SEQ ID NO:5 Light Chain Variable Region (VL) Amino Acid Sequence; Anti-CD26 mAb (begelomab)

QIVLTQSPAIMSASPGEKVTITCSASSSVSYMNWFQQKPGTSPKLWIYST
SNLASGVPARFSGSGSGTSYSLTISRMEAEDAATYYCQQRSSYPNTFGGG
TKLEIK

SEQ ID NO:6 Light Chain Variable Region (VL) Nucleotide Sequence; Anti-CD26 mAb (begelomab)

Caaattgttctcacccagtctccagcaatcatgtctgcatctccagggga
gaaggtcaccataacctgcagtgccagctcaagtgtaagttacatgaact
ggttccagcagaagccaggcacttctcccaaactctggatttatagcacc
tccaacctggcttctggagtccctgctcgcttcagtggcagtggatctgg
gacctcttactctctcacaatcagccgaatggaggctgaagatgctgcca
cttattactgccagcaaaggagtagttacccgaacacgttcggagggggg
accaagctggaaataaaa

SEQ ID NO:7 Heavy Chain CDR1 Amino Acid Sequence; Anti-CD26 mAb (begelomab)

GYTFRSYDIN

SEQ ID NO:8 Heavy Chain CDR2 Amino Acid Sequence; Anti-CD26 mAb (begelomab)

WIFPGDGSTKYNEKFK

SEQ ID NO:9 Heavy Chain CDR3 Amino Acid Sequence; Anti-CD26 mAb (begelomab)

WTVVGPGYFDV

SEQ ID NO:10 Light Chain CDR1 Amino Acid Sequence; Anti-CD26 mAb (begelomab)

SASSSVSYMN

SEQ ID NO:11 Light Chain CDR2 Amino Acid Sequence; Anti-CD26 mAb (begelomab)

STSNLAS

SEQ ID NO:12 Light Chain CDR3 Amino Acid Sequence; Anti-CD26 mAb (begelomab)

QQRSSYPNT

SEQ ID NO:13 Heavy Chain Constant Region Nucleotide Sequence; Anti-CD26 mAb (begelomab)

gccaaaacaacacccccatcagtctatccactggcccctgggtgtggaga
tacaactggttcctccgtgactctgggatgcctggtcaagggctacttcc
ctgagtcagtgactgtgacttggaactctggatccctgtccagcagtgtg
cacaccttcccagctctcctgcagtctggactctacactatgagcagctc
agtgactgtcccctccagcacctggccaagtcagaccgtcacctgcagcg
ttgctcacccagccagcagcaccacggtggacaaaaaacttgagcccagc
gggcccatttcaacaatcaacccctgtcctccatgcaaggagtgtcacaa
atgcccagctcctaacctcgagggtggaccatccgtcttcatcttccctc
caaatatcaaggatgtactcatgatctccctgacacccaaggtcacgtgt
gtggtggtggatgtgagcgaggatgacccagacgtccagatcagctggtt
tgtgaacaacgtggaagtacacacagctcagacacaaacccatagagagg
attacaacagtactatccgggtggtcagcaccctccccatccagcaccag
gactggatgagtggcaaggagttcaaatgcaaggtcaacaacaaagacct
cccatcacccatcgagagaaccatctcaaaaattaaagggctagtcagag
ctccacaagtatacatcttgccgccaccagcagagcagttgtccaggaaa
gatgtcagtctcacttgcctggtcgtgggcttcaaccctggagacatcag
tgtggagtggaccagcaatgggcatacagaggagaactacaaggacaccg
caccagtcctggactctgacggttcttacttcatatatagcaagctcaat
atgaaaacaagcaagtgggagaaaacagattccttctcatgcaacgtgag
acacgagggtctgaaaaattactacctgaagaagaccatctcccggtctc
cgggtaaa

SEQ ID NO:14 Light Chain Constant Nucleotide Sequence; Anti-CD26 mAb (begelomab)

cgggctgatgctgcaccaactgtatccatcttcccaccatccagtgagca
gttaacatctggaggtgcctcagtcgtgtgcttcttgaacaacttctacc
ccaaagacatcaatgtcaagtggaagattgatggcagtgaacgacaaaat
ggcgtcctgaacagttggactgatcaggacagcaaagacagcacctacag
catgagcagcaccctcacgttgaccaaggacgagtatgaacgacataaca
gttatacctgtgaggccactcacaagacatcaacttcacccattgtcaag
agcttcaacaggaatgagtgt

SEQ ID NO:15 Heavy Chain Nucleotide Sequence; Anti-CD26 mAb (begelomab-CHO optimized)

caagtgcagcttcagcagtccggagccgaactcgtgaagccgggagcttc
cgtgaagctgagctgcaaggcatcggggtataccttccgctcctacgaca
tcaactgggtcagacagaggcccgaacagggtctggaatggattggctgg
atcttccctggcgacggttccaccaagtacaacgagaagttcaagggcaa
agccaccctgaccactgacaagtcctcatcgaccgcgtacatgcaattga
gccggctgacctccgaggatagcgccgtgtacttctgtgcccggtggact
gtcgtgggaccaggctactttgatgtctggggggccggaactaccgtgac
ggtgtcatcagccaagactacccctccgtccgtgtacccccttgctccgg
gatgtggagacaccaccggctcgtccgtcactctgggatgcctcgtgaag
ggatacttccccgaatccgtcaccgtgacctggaacagcggaagcctgtc
ctcgtccgtgcatactttccctgccctgctgcaatccggcctgtacacca
tgagctccagcgtgaccgtgccatcctcgacctggcccagccagaccgtg
acttgctcagtggcgcaccctgcctcatccactaccgtggacaagaagcc
gagccctccggtccgatttcaaccatcaacccttgcccaccctgcaaaga
atgccataagtgtcccgctccgaatctagaaggcggcccatccgtcttta
tcttccctcccaacattaaggacgtgctgatgattagtctgaccccgaaa
gtcacttgcgtggtggtggacgtgtccgaagatgacccagacggcagatc
tcatggttcgtgaacaacgtggaggtgcacacggcccagacccagacgca
ccgggaggactacaactcgactatccgcgtggtgtccacccttccgatcc
aacaccaggattggatgtcggggaaggagttcaagtgcaaggtcaacaac
aaggatctcccgtcccccattgagaggacaatctctaagatcaagggcct
cgtcagagcgcctcaggtctacatcttgcctcctcccgccgaacagttga
gccggaaggatgtgtccctgacttgtctggtcgtggggttcaatccggga
gacatctccgtggagtggacctcgaacggacacaccgaggaaaactacaa
ggacactgcaccggtgctggattccgacggctcctatttcatctactcga
agctgaacatgaaaacctcgaaatgggaaaagactgacagcttcagctgc
aacgtgcgccacgagggtctgaagaactactacctgaaaaagaccatttc
acggtccccggggaaa

SEQ ID NO:16 Light Chain Nucleotide Sequence; Anti-CD26 mAb (begelomab-CHO optimized)

Cagatcgtgcttacccaatccccggcgattatgtcagccagccccggaga
aaaggtcaccattacttgctcggcatcctcctccgtgtcgtacatgaact
ggttccagcaaaagcccggcactagcccaaagctgtggatctattccacg
tccaacctggcgtcaggagtgcctgcccgcttttcgggttctggcagcgg
gactagctactccctcaccatctcgagaatggaagctgaggacgccgcca
cctactactgtcagcagcggtcctcctacccgaacaccttcgggggaggc
accaaactggagatcaaacgggctgatgctgcaccaactgtatccatctt
cccaccatccagtgagcagttaacatctggaggtgcctcagtcgtgtgct
tcttgaacaacttctaccccaaagacatcaatgtcaagtggaagattgat
ggcagtgaacgacaaaatggcgtcctgaacagttggactgatcaggacag
caaagacagcacctacagcatgagcagcaccctcacgttgaccaaggacg
agtatgaacgacataacagctatacctgtgaggccactcacaagacatca
acttcacccatcgtcaagagcttcaacaggaatgagtgt

What is claimed is:

1. A method of inhibiting inflammation in a human patient having an idiopathic inflammatory myopathy (IIM), the method comprising administering to the patient an effective amount of an antibody that specifically binds CD26; wherein the anti-CD26 antibody comprises (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO: 12.

2. A method of treating an idiopathic inflammatory myopathy (IIM), the method comprising administering to the patient an effective amount of an antibody that specifically binds CD26; wherein the anti-CD26 antibody comprises (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO: 12.

3. The method of claim 1, wherein the IIM is dermatomyositis (DM), polymyositis (PM), necrotizing myopathy (NM), sporadic inclusion body myositis (IBM), or overlap myositis (OM).

4. The method of claim 1, wherein the anti-CD26 antibody is a full-length antibody.

5. The method of claim 1, wherein the antibody is a monoclonal, human, humanized, chimeric, multivalent antibody, or an antigen-binding fragment thereof.

6. (canceled)

7. The method of claim 1, wherein the antibody has an IgG2b isotype.

8. The method of claim 1, wherein the anti-CD26 antibody is begelomab, 1F7, or CM03.

9. The method of claim 8, wherein the anti-CD26 antibody is produced in Chinese hamster ovary (CHO) cells.

10. The method of any claim 1, wherein the anti-CD26 antibody is produced from a hybridoma cell line deposited at CBA-ICLC of Genoa (Italy) as deposit number PD 12002.

11. (canceled)

12. The method of claim 1, wherein the anti-CD26 antibody comprises heavy and light chain variable regions comprising the sequences set forth in SEQ ID NOs:3 and 5, respectively.

13. The method of claim 1, wherein the anti-CD26 antibody comprises heavy and light chains constant regions comprising the sequences set forth in SEQ ID NOs: 1 and 2, respectively.

14. The method of claim 1, further comprising administration of an immunosuppressive agent.

15. The method of claim 14, wherein the immunosuppressive agent is a corticosteroid, methotrexate, azathioprine, mycophenolate mofetil, methylprednisone, cyclophosphamide, cyclosporineA, tacrolimus, or a combination thereof.

16. The method of claim 14, wherein the immunosuppressive agent is given prior to, concomitantly with, or after the anti-CD26 antibody.

17. The method of claim 1, wherein the cycle is a period of 4 or 5 weeks, and wherein for each of the at least one cycle, 16 doses of the anti-CD26 antibody are administered at a dose of between 4.0 mg/m² to 25 mg/m².

18. The method of claim 1, wherein the anti-CD26 antibody is administered at a dose of 4, 5, 6, 7, 8, 9, 10, 11, 12, 13, 14, 15, 16, 17, 18, 19, 20, 21, 22, 23, 24, or 25 mg/m².

19. The method of claim 1, wherein a fixed dose of the anti-CD26 antibody is administered.

20. The method of claim 19, wherein the fixed dose is determined based on the immunosuppressive agent administered to the subject.

21. A method of inhibiting inflammation in a human patient having an idiopathic inflammatory myopathy (IIM), the method comprising administering to the patient an effective amount of an anti-CD26 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, wherein the patient’s CD3+ T cells and/or CD31+ endothelial cells express CD26.

22. A method of treating an idiopathic inflammatory myopathy (IIM) in a human patient, the method comprising administering to the patient an effective amount of an anti-CD26 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, wherein the patient’s CD3+ T cells and/or CD31+ endothelial cells express CD26.

23. The method of claim 21, wherein the method is administered to a patient that has not received prior therapy (e.g., first line therapy).

24. (canceled)

25. The method of claim 21, wherein expression of CD26 is assayed by RT-PCR, in situ hybridization, RNase protection, RT-PCR-based assay, immunohistochemistry, enzyme linked immuosorbent assay, in vivo imaging, or flow cytometry.

26. (canceled)

27. An antibody that specifically binds CD26 comprising the heavy chain and light chain nucleotide sequences comprising SEQ ID NO: 15 and SEQ ID NO: 16, respectively.

28-29. (canceled)


SEQUENCE LISTING (16 sequences)

SEQ ID NO: 1 - protein, 336 aa, Artificial Sequence
  Heavy Chain Constant Region Amino Acid Sequence; Anti-CD26 mAb (begelomab)
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 2 - protein, 107 aa, Artificial Sequence
  Light Chain Constant Region Amino Acid Sequence; Anti-CD26 mAb (begelomab)
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 120 aa, Artificial Sequence
  Heavy Chain Variable Region (VH) Amino Acid Sequence; Anti-CD26 mAb (begelomab)
QVQLQQSGAELVKPGASVKLSCKASGYTFRSYDINWVRQRPEQGLEWIGWIFPGDGSTKYNEKFKGKATLTTDKSSSTAYMQLSRLTSEDSAVYFCARWTVVGPGYFDVWGAGTTVTVSS

SEQ ID NO: 4 - DNA, 360 bp, Artificial Sequence
  Heavy Chain Variable Region (VH) Nucleotide Sequence; Anti-CD26 mAb (begelomab)
caggtccagctgcagcagtctggagctgaactggtaaagcctggggcttcagtgaagttgtcctgcaaggcttctggctacaccttcagaagttatgatataaactgggtgagacagaggcctgaacagggacttgagtggattggatggatttttcctggagatggtagtactaagtacaatgagaagttcaagggcaaggccacactgactacagacaaatcctccagcacagcctacatgcagctcagcaggctgacatctgaggactctgctgtctatttctgtgcaagatggacggtagtaggcccagggtacttcgatgtctggggcgcagggaccacggtcaccgtctcctca

SEQ ID NO: 5 - protein, 106 aa, Artificial Sequence
  Light Chain Variable Region (VL) Amino Acid Sequence; Anti-CD26 mAb (begelomab)
QIVLTQSPAIMSASPGEKVTITCSASSSVSYMNWFQQKPGTSPKLWIYSTSNLASGVPARFSGSGSGTSYSLTISRMEAEDAATYYCQQRSSYPNTFGGGTKLEIK

SEQ ID NO: 6 - DNA, 318 bp, Artificial Sequence
  Light Chain Variable Region (VL) Nucleotide Sequence; Anti-CD26 mAb (begelomab)
caaattgttctcacccagtctccagcaatcatgtctgcatctccaggggagaaggtcaccataacctgcagtgccagctcaagtgtaagttacatgaactggttccagcagaagccaggcacttctcccaaactctggatttatagcacctccaacctggcttctggagtccctgctcgcttcagtggcagtggatctgggacctcttactctctcacaatcagccgaatggaggctgaagatgctgccacttattactgccagcaaaggagtagttacccgaacacgttcggaggggggaccaagctggaaataaaa

SEQ ID NO: 7 - protein, 10 aa, Artificial Sequence
  Heavy Chain CDR1 Amino Acid Sequence; Anti-CD26 mAb (begelomab)
GYTFRSYDIN

SEQ ID NO: 8 - protein, 16 aa, Artificial Sequence
  Heavy Chain CDR2 Amino Acid Sequence; Anti-CD26 mAb (begelomab)
WIFPGDGSTKYNEKFK

SEQ ID NO: 9 - protein, 11 aa, Artificial Sequence
  Heavy Chain CDR3 Amino Acid Sequence; Anti-CD26 mAb (begelomab)
WTVVGPGYFDV

SEQ ID NO: 10 - protein, 10 aa, Artificial Sequence
  Light Chain CDR1 Amino Acid Sequence; Anti-CD26 mAb (begelomab)
SASSSVSYMN

SEQ ID NO: 11 - protein, 7 aa, Artificial Sequence
  Light Chain CDR2 Amino Acid Sequence; Anti-CD26 mAb (begelomab)
STSNLAS

SEQ ID NO: 12 - protein, 9 aa, Artificial Sequence
  Light Chain CDR3 Amino Acid Sequence; Anti-CD26 mAb (begelomab)
QQRSSYPNT

SEQ ID NO: 13 - DNA, 1008 bp, Artificial Sequence
  Heavy Chain Constant Region Nucleotide Sequence; Anti-CD26 mAb (begelomab)
gccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 14 - DNA, 321 bp, Artificial Sequence
  Light Chain Constant Nucleotide Sequence; Anti-CD26 mAb (begelomab)
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagttatacctgtgaggccactcacaagacatcaacttcacccattgtcaagagcttcaacaggaatgagtgt

SEQ ID NO: 15 - DNA, 1368 bp, Artificial Sequence
  Heavy Chain Nucleotide Sequence; Anti-CD26 mAb (begelomab-CHO optimized)
caagtgcagcttcagcagtccggagccgaactcgtgaagccgggagcttccgtgaagctgagctgcaaggcatcggggtataccttccgctcctacgacatcaactgggtcagacagaggcccgaacagggtctggaatggattggctggatcttccctggcgacggttccaccaagtacaacgagaagttcaagggcaaagccaccctgaccactgacaagtcctcatcgaccgcgtacatgcaattgagccggctgacctccgaggatagcgccgtgtacttctgtgcccggtggactgtcgtgggaccaggctactttgatgtctggggggccggaactaccgtgacggtgtcatcagccaagactacccctccgtccgtgtacccccttgctccgggatgtggagacaccaccggctcgtccgtcactctgggatgcctcgtgaagggatacttccccgaatccgtcaccgtgacctggaacagcggaagcctgtcctcgtccgtgcatactttccctgccctgctgcaatccggcctgtacaccatgagctccagcgtgaccgtgccatcctcgacctggcccagccagaccgtgacttgctcagtggcgcaccctgcctcatccactaccgtggacaagaagctcgagccctccggtccgatttcaaccatcaacccttgcccaccctgcaaagaatgccataagtgtcccgctccgaatctagaaggcggcccatccgtctttatcttccctcccaacattaaggacgtgctgatgattagtctgaccccgaaagtcacttgcgtggtggtggacgtgtccgaagatgacccagacgtgcagatctcatggttcgtgaacaacgtggaggtgcacacggcccagacccagacgcaccgggaggactacaactcgactatccgcgtggtgtccacccttccgatccaacaccaggattggatgtcggggaaggagttcaagtgcaaggtcaacaacaaggatctcccgtcccccattgagaggacaatctctaagatcaagggcctcgtcagagcgcctcaggtctacatcttgcctcctcccgccgaacagttgagccggaaggatgtgtccctgacttgtctggtcgtggggttcaatccgggagacatctccgtggagtggacctcgaacggacacaccgaggaaaactacaaggacactgcaccggtgctggattccgacggctcctatttcatctactcgaagctgaacatgaaaacctcgaaatgggaaaagactgacagcttcagctgcaacgtgcgccacgagggtctgaagaactactacctgaaaaagaccatttcacggtccccggggaaa

SEQ ID NO: 16 - DNA, 639 bp, Artificial Sequence
  Light Chain Nucleotide Sequence; Anti-CD26 mAb (begelomab-CHO optimized)
cagatcgtgcttacccaatccccggcgattatgtcagccagccccggagaaaaggtcaccattacttgctcggcatcctcctccgtgtcgtacatgaactggttccagcaaaagcccggcactagcccaaagctgtggatctattccacgtccaacctggcgtcaggagtgcctgcccgcttttcgggttctggcagcgggactagctactccctcaccatctcgagaatggaagctgaggacgccgccacctactactgtcagcagcggtcctcctacccgaacaccttcgggggaggcaccaaactggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt